(19) United States
(12) Patent Application Publication
(10) Pub. No.: US 2026/0328225 A1
(43) Pub. Date: Oct. 22, 2026

(54) VOLUTE FOR A TURBOMACHINE

(71) Applicant: Dyson Technology Limited, Malmesbury, Wiltshire (GB)

(72) Inventors: Thiago Rubens VIEIRA EBEL, Swindon (GB); Christopher Ashley MONK, Royal Wootton Bassett (GB); Ryan Christopher BENSON, Swindon (GB); Scott VAN DER LEEDEN, Bristol (GB)

(21) Appl. No.: 19/107,644

(22) PCT Filed: Aug. 16, 2023
(86) PCT No.: PCT/IB2023/058216

§ 371 Date: Feb. 28, 2025

(30) Foreign Application Priority Data
Aug. 31, 2022 (GB) 2212647.8

(51) Int. Cl. F04D 29/66; A62B 18/00; F04D 17/10; F04D 25/08; F04D 29/42

(52) U.S. Cl. CPC F04D 29/664; A62B 18/006; F04D 17/10; F04D 25/08; F04D 29/4233; F05D 2300/514

(57) ABSTRACT

Provided is a volute for a turbomachine. The volute includes a scroll, a volute exit, and a tongue between the scroll and the volute exit. The tongue includes a porous material that is attached to a wall of the volute. Also provided is a turbomachine incorporating the volute, a wearable air purifier incorporating the turbomachine and a method of forming the volute.

### TECHNICAL FIELD

The present disclosure relates to a volute for a turbomachine. The volute finds particular, but not exclusive, use in wearable devices (e.g. wearable air purifiers) and home appliances (e.g. fans).

### BACKGROUND

Some turbomachines (such as a centrifugal compressors) include an impeller that is circumferentially surrounded by a volute. In use, fluid enters an inlet of the turbomachine and is driven radially outwardly by the impeller into a scroll of the volute. Fluid received in the scroll flows along the scroll to a volute exit where it is discharged from the turbomachine.

In such turbomachines, the volute includes a volute tongue (also referred to as a cutwater) that separates the scroll from the volute exit. In particular, the volute tongue is positioned at an entrance to the volute exit and divides the fluid into a first stream that enters the volute exit and a second stream that continues to circulate in the scroll, about the impeller.

Conventional thinking in the field is that the volute tongue should be configured to interact with fluid in a manner that means the volute tongue imparts minimal resistance and turbulence to the fluid. Conventional volute tongues thus usually have a smooth leading face aligned with the fluid direction to minimise separation and de-attachment of fluid across the volute.

In operation, the movement of the impeller and its blades generate pressure fluctuations which cause acoustic waves (noise) from turbulence (causing broadband noise) and other factors such as interactions of moving and stationary parts (generating particular tones). In many applications of a turbomachine there is a general desire to minimise acoustic noise. In some applications, however, there is increased incentive to reduce such acoustic noise because it can be particularly detrimental to user's experience. One example is a wearable air purifier, which is worn on the head of the user and includes a turbomachine (e.g. a centrifugal compressor) that supplies purified air to a user's mouth. The proximity of the turbomachine to a user's ears, due to the device being worn on the user's head, means that the suppression of noise is especially important for this type of device.

The present disclosure has been devised in light of the above considerations.

### SUMMARY

In a first aspect there is provided a volute for a turbomachine, the volute comprising a scroll, a volute exit, and a tongue between the scroll and the volute exit, the tongue comprising porous material that is attached to a wall of the volute.

Providing a volute tongue that comprises a porous material can aid in suppressing acoustic noise produced during operation of a turbomachine incorporating the volute. One particular source of such acoustic noise in turbomachines is the interaction between the volute tongue and fluid departing from the blades of an impeller. This acoustic noise forms a tone, causing a characteristic acoustic ‘ringing’ which is proportional to the impeller rotational speed and numbers of blades. Humans are particularly perceptive to the tone generated by impeller speeds between e.g. 4,000 rpm and 20,000 rpm and impeller blade numbers between e.g. 3 and 25 blades. The porous material can distort turbulence in the fluid that encounters the tongue so as to reduce the generation of acoustic noise.

Further, as the porous material is attached (i.e. directly) to a wall of the volute (referred to herein as the “volute wall”), it does not require containment within a separate containing structure which would otherwise be required to retain the porous material on the tongue. This simplifies the structure of the volute so that it is easier and more cost effective to manufacture and package. Additionally, in the absence of a containing structure, the porous material may be directly exposed to incident fluid, which avoids possible interference by a containing structure. Such interference could be detrimental to the acoustic noise suppression performance of the porous material.

Optional features of the first aspect will now be set out. These are applicable singly or in any combination with any aspect.

The porous material may have a three-dimensional porous structure (as opposed to a two-dimensional porous structure, such as a perforated sheet or mesh). For example, the porous material may comprise a network of interconnected pores (the pores may be formed according to a regular or irregular pattern). Each pore of the porous material may have a length (being the amount it extends into the porous material) that is greater than its diameter or width, taken perpendicular to the length. One or more pores of the porous structure may change direction along their length (i.e. may have a bend). The three-dimensional nature of the porous structure may provide improved acoustic noise suppression.

The porous material may be infrangible. The porous material may therefore be configured to hold itself together so as not to break apart in normal use due to incident fluid flow. Providing an infrangible porous material may avoid the need to provide a separate containing structure to contain the porous material. A frangible porous material, such as a loose fibre material, would break apart under incident fluid flow, and would therefore require containment within a containing structure to achieve an acceptable service life.

The porous material may be self-supporting, so that no further containing (or supporting) structure needs to be provided to prevent the porous material from breaking apart in normal use due to incident fluid flow.

The porous material may, for example, comprise felt or foamed plastic.

The porous material may have an open cell structure. An open cell structure may provide better acoustic noise suppression over a closed cell structure.

The porous material may define an external surface of the volute tongue. The external surface may be referred to as a “wetted surface” of the volute tongue (i.e. a surface that is in contact with the main flow of fluid passing through the volute). For the avoidance of doubt, surfaces formed within the porous material by the internal porous structure of the porous material are not considered to be “external surfaces” because they are internal to the porous material when taken as a whole. Rather, the external surface is considered to be that which defines the external boundary or envelope of the volute tongue.

The porous material may form a leading edge of the tongue (i.e. the free end or edge of the tongue that fluid first comes into contact with as it flows across the tongue). The leading edge of the tongue may be particularly susceptible to the generation of acoustic noise because this is the surface that splits the fluid flow between the volute exit and the scroll. The provision of porous material at the leading edge may therefore be particularly effective in terms of suppressing acoustic noise.

The volute wall to which the porous material is attached, may be non-porous (i.e. at least in proximity to the porous material). A non-porous wall may function better as an aerodynamic surface for fluid flow than a porous wall. Thus, the combination of a non-porous volute wall and the porous material of the tongue may provide a suitable balance between acoustic noise suppression and efficient flow of fluid.

The volute wall may divide the scroll and the volute exit. The porous material may extend along a side of the wall. The extension of the porous material along a side of the volute wall may increase the contact area between the porous material and the volute wall, which in turn may provide stronger attachment between the porous material and the volute wall. Further, the volute wall may at least partly restrict flow (i.e. leakage) of fluid across the porous material in a direction extending between the scroll and the volute exit. Such leakage could otherwise result in undesirable turbulence in the volute exit.

The porous material may comprise an impeller-facing (or scroll-facing) portion extending along a first side of the volute wall. The impeller-facing portion of the porous material may be exposed to fluid circulating in the scroll in use.

The porous material may comprise a volute exit-facing portion extending along a second side of the volute wall that is opposite to the first side of the volute wall. The volute exit-facing portion of the porous material may be exposed to fluid in the volute exit in use.

The impeller-facing and volute-exit facing portions may be joined (e.g. at the leading edge of the tongue). Hence, the porous material may extend about a leading edge of the volute wall and continuously therefrom along the opposite first and second sides of the volute wall. That is, the volute wall may extend into the porous material (and may be sandwiched between the impeller-facing and volute-exit facing portions).

An outer surface (i.e. the wetted surface) of the porous material may be convex. The outer surface may be shaped so as to form a continuous (i.e. flush) surface with adjacent regions of the wetted surface of the volute wall (i.e. a smooth transition may be provided between the outer surface of the porous material and the wetted surface of the volute wall).

The porous material may extend for substantially the entire width of the volute tongue (i.e. in a direction transverse to the direction of fluid flow in normal use of the volute).

Each of the volute wall (to which the porous material is attached) and the porous material may comprise one of a male portion (e.g. protrusion) and female portion (e.g. recess). The male and female portions may be mated to attach the porous material to the volute wall. For example, the volute wall may define a recess and the porous material may be at least partly located in the recess. The porous material and/or the male portion may comprise location pins (e.g. receivable in location recesses formed in the other of the porous material and male portion). Such location pins may avoid misalignment of the porous material.

The volute may comprise first and second volute parts, e.g, halves, that are joined together. The porous material may be at least partially sandwiched between the first and second volute parts (i.e. at least part of the porous material being retained in a recess defined between the volute halves). Thus, the porous material may be compressed between the first and second volute halves. One or both volute halves may comprise means (such as a surface treatment, coating, texture or layer) configured to grip the porous material (i.e. so as to increase friction between the porous material and the volute halves).

The porous material may be attached to the volute wall by one or more of an interference fit, a mechanical fixture (e.g. bolt, screw, snap-engagement, etc.), adhesive, or a weld (e.g. ultrasonic weld). The porous material may be integral with the volute wall (together, the volute wall and the porous material may form a monolithic structure).

In a second aspect there is provided a turbomachine comprising an impeller, a motor (e.g. an electric motor) operable to rotate the impeller, and a volute according to the first aspect, for collecting fluid expelled by the impeller.

The turbomachine may be a rotodynamic compressor (e.g. a centrifugal compressor, axial compressor or mixed-flow compressor).

The turbomachine may be configured to operate with a blade pass frequency of 50 Hz to 11,000 Hz, or e.g. 800 Hz to 8000 Hz.

In a third aspect, there is provided a wearable air purifier comprising a turbomachine according to the second aspect, headgear for mounting on a wearer's head to attach the turbomachine to a wearer's head, and a duct for directing fluid discharged by the turbomachine towards a wearer's face when the headgear is mounted on the wearer's head.

The wearable air purifier may comprise an air treatment device arranged to treat air upstream or downstream of the turbomachine.

In a fourth aspect, there is provided a method of forming a volute for a turbomachine, the method comprising providing a volute precursor comprising a scroll and a volute exit, and attaching porous material to a volute wall precursor to form a tongue between the scroll and the volute exit.

The step of attaching the porous material to the volute wall may comprise at least one of injection moulding, applying adhesive to the volute wall and/or porous material, and welding (e.g. ultrasonic welding) the porous material to the volute wall.

The injection moulding may comprise over-moulding the porous material onto the volute wall (e.g. onto a male portion of the volute wall). The injection moulding may comprise twin-shot moulding the porous material with the volute wall.

In some embodiments, the volute precursor may comprise first and second volute precursor halves that are joinable together. The step of attaching the porous material to the volute wall may comprise positioning at least part of the porous material between the first and second volute precursor halves and joining the volute precursor halves together to retain the porous material therebetween (i.e. to sandwich the porous material between the volute precursor halves).

The method may comprise forming the volute precursor (or volute precursor halves) by injection moulding.

The volute (e.g. the volute wall and the porous material) formed by the method of the fourth aspect may be as described above with respect to the first aspect.

In a fifth aspect, there is provided a kit of parts for building a turbomachine, the kit comprising:
an impeller;
a motor (e.g. electric motor) operable to rotate the impeller; and
a volute according to the first aspect, for collecting fluid expelled by the impeller.

### BRIEF SUMMARY OF THE FIGURES

Embodiments will now be discussed with reference to the accompanying figures in which:

FIG. 1 is a perspective cutaway view of a turbomachine according to a first embodiment;

FIG. 2 is a front section view of a volute tongue of the turbomachine of the first embodiment;

FIG. 3 is a top section view of the volute tongue of the turbomachine of the first embodiment;

FIG. 4 is a top section view of a volute tongue of a turbomachine according to a second embodiment; and

FIG. 5 is a top section view of a volute tongue of a turbomachine according to a third embodiment.

### DETAILED DESCRIPTION

Aspects and embodiments will now be discussed with reference to the accompanying figures. Further aspects and embodiments will be apparent to those skilled in the art.

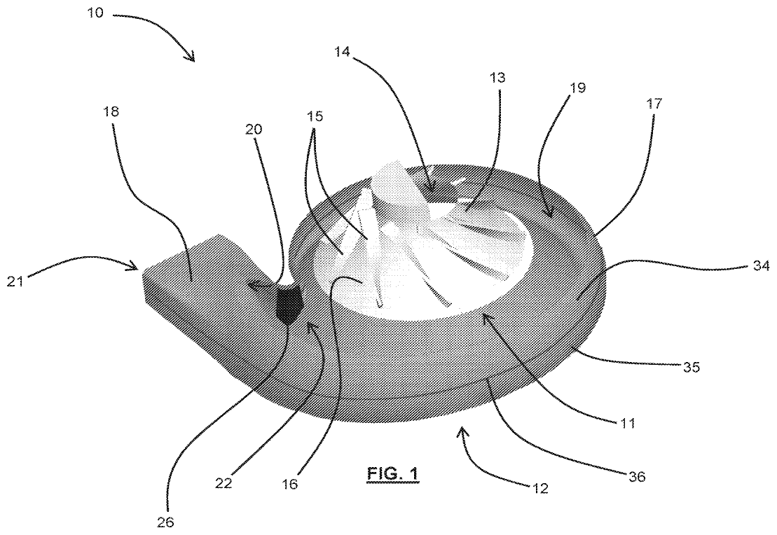

FIG. 1A illustrates a turbomachine in the form of a centrifugal compressor 10. The compressor 10 includes an impeller 11 that is circumferentially surrounded by a volute 12. The impeller 11 comprises a body 13 and blades 15 that project radially outwardly from an outer frustoconical surface 16 of the body 13. The body 13 comprises a central aperture (at the narrower end of the frustoconical outer surface 16) that defines an inlet 14, through which fluid flows into the compressor 10. Air is drawn through the inlet 14 by the impeller 11, which rotates about a rotational axis that defines an axial direction of the compressor 10. The illustrated impeller 11 is configured for clockwise rotation, but in other embodiments the impeller may be configured for anti-clockwise rotation.

Although not shown, the compressor 10 is also provided with a compressor housing that encloses front and rear sides (upper and lower as illustrated) of the impeller 11, but which accommodates the inlet 14 and includes a circumferential opening surrounding the impeller 11 to permit radial flow of fluid from the impeller 11 to the volute 12. One purpose of the impeller housing is that it provides means for sealingly mounting the impeller 11 within the central opening of the volute 12.

The volute 12 comprises a spiral-shaped scroll 17 and a volute exit 18 that extends tangentially from the scroll 17. The scroll 17 comprises a scroll chamber 19 that is open on a radially inward face thereof for receipt of fluid from the impeller 11. A cross-sectional area of the scroll chamber 19 (i.e. taken in the radial direction) increases gradually in a direction of fluid flow (clockwise, as illustrated) along the chamber from an upstream end thereof to a downstream end adjacent to the volute exit 18.

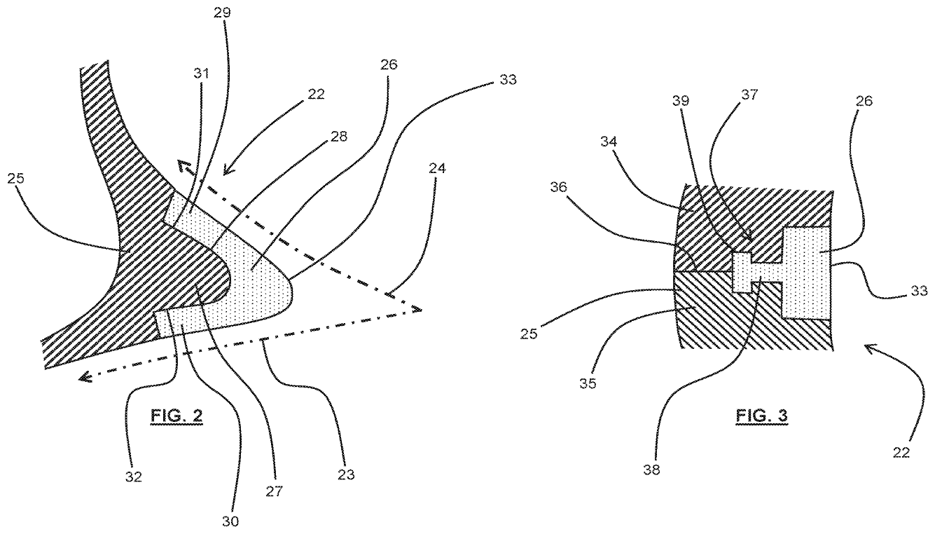

The volute exit 18 comprises a volute exit passage 20 through which fluid flows from the downstream end of the scroll chamber 19 to an outlet 21 of the compressor 10. A volute tongue 22 is provided at an entrance to the volute exit passage 20, between the volute exit 18 and the scroll 17. The volute tongue 22 extends in an axial direction (i.e. parallel to the rotational axis of the impeller) across the volute 12. In this way, the volute tongue 22 divides fluid circulating in the scroll chamber 19 into a first stream 23 that enters the volute exit 18 (and is discharged from the compressor 10) and a second stream 24 that continues to circulate in the scroll chamber 19 (the streams 23, 24 are shown in FIG. 2, which is discussed in more detail further below).

In operation, fluid (in this case air) enters the compressor 10 axially through the inlet 14 and is driven radially outwardly and into the scroll chamber 19 by rotation of the impeller 11. Fluid received in the scroll chamber 19 flows along the scroll chamber 19 towards the volute exit 18 where a portion of the fluid is diverted to the volute exit 18 by the volute tongue 22. This portion of the fluid is then discharged from the compressor 10 through the outlet 21.

The volute tongue 22 is shown in more detail in FIGS. 2 and 3. As is apparent from these figures, the volute tongue 22 comprises a wall 25 of the volute 12 and a porous material 26 that is attached to the wall 25. Although not apparent from the figures, the porous material 26 may comprise felt, the fibres of which form a three-dimensional porous structure. However, another option for the porous material 26 is to form it from foamed plastic, e.g. having an open cell structure. Preferably, the porous material 26 is infrangible and holds itself together so as not to break apart in normal use due to incident fluid flow. As has already been discussed, providing the volute tongue 22 with a porous material 26 can aid in suppression of acoustic noise in the compressor 10. In particular, the noise suppressing ability of the porous material 26 is enhanced by directly exposing it to the incident fluid flow and not shielding it behind any kind of containment structure.

The wall 25 of the volute comprises an axially extending lip 27 (i.e. the lip 27 extends across the volute 12 in a direction that is parallel to the rotational axis of the impeller 11). The lip 27 has an outer convex surface 28 over which the porous material 26 is wrapped, such that the porous material 26 has a U-shaped profile that is complementary in shape to the outer convex surface 28. In other words, the porous material 26 is shaped so as to define a leading edge of the volute tongue 22 and an elongate axially extending recess in which the lip 27 is received.

This arrangement of the porous material 26 on the volute wall 25 means that the porous material 26 includes an impeller-facing portion 29 that extends along a first (impeller-facing) side 31 of the lip 27 (the upper side as illustrated). The porous material 26 also includes a volute exit-facing portion 30 that extends along a second side 32 of the lip 27. The lip 27 is thus located between the impeller-facing 29 and volute exit-facing 30 portions so as to present an obstruction to fluid flowing through the porous material 26, between the volute exit passage 20 and the scroll chamber 19.

As is apparent from FIG. 2, an outer (external) surface 33 of the porous material 26 forms a continuous surface with adjacent surfaces of the volute wall 25 (i.e. the outer surface 33 is flush with adjacent regions of the volute wall 25). This is achieved by the provision of the recessed region in the volute wall 25 (in which the porous material 26 is received) that is complementary in shape to the porous material 26.

FIG. 3 illustrates how the porous material 26 of the illustrated embodiment is attached to the volute wall 25. As is apparent from this figure, the volute 12 is formed of front 34 and rear 35 volute halves joined along a split line 36. The volute halves 34, 35 are configured such that, when joined in this manner, they form a female portion in the form of a cavity 37. The porous material 26 includes a corresponding male portion in the form of a protrusion 38 that protrudes into the cavity 37, to be clamped within the cavity 37, between the first 34 and second 35 volute halves. The protrusion 38 includes ribs 39 that anchor the protrusion 38 within the cavity 37 so as to prevent the porous material 26 from being dislodged in use. In this way, the porous material 26 is mated with the volute wall 25 without requiring additional means such as adhesive or ultrasonic welding.

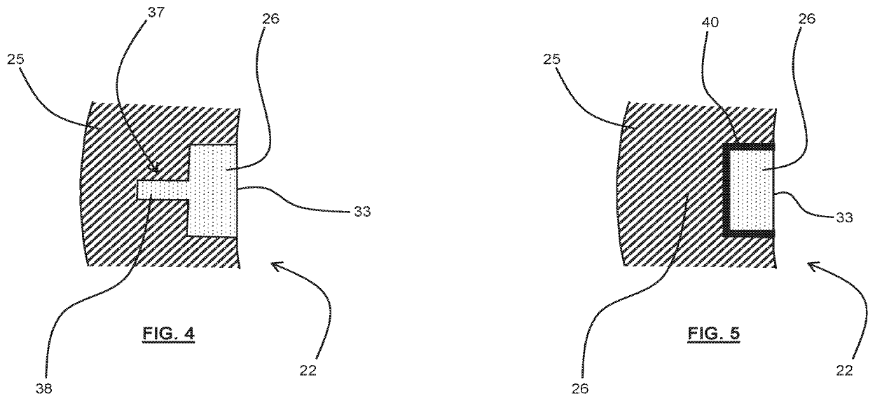

FIG. 4 illustrates a second embodiment that is a variation of the first embodiment described above (and shown in FIGS. 1 to 3). Given many of the features remain the same, the same reference numerals are used. Only differences are discussed below.

The embodiment of FIG. 4 differs in the way the porous material 26 is attached to the volute wall 25. In this embodiment, the volute 12 is formed of a single part (as opposed to the previous embodiment, which included a volute 12 formed of two halves). The porous material 26 includes a protrusion 38 that protrudes into a cavity 37 formed in the volute wall 25. The protrusion 38 and cavity 37 are sized so as to provide an interference fit which retains the protrusion 38 within the cavity 37 (and thus the porous material 26 to the volute wall 25). As may be appreciated, the protrusion 38 may be additionally or alternatively retained in the cavity 37 by way of an adhesive, or by being moulded with (e.g. twin-shot or over moulding) the volute wall 25.

FIG. 5 illustrates a third embodiment that represents a further variation of the previously described embodiments (again, the same reference numerals are used). In this embodiment, the volute 12 is, again, formed of a single part. The porous material 26 does not include a protrusion and, likewise, the volute wall 25 does not include a corresponding cavity for receipt of such a protrusion. Instead, the porous material 26 is attached to the volute wall 25 by an adhesive layer 40 provided at the boundary between the volute wall 25 and the porous material 26. Of course, the porous material 26 could otherwise be attached by way of a mechanical fixture, welding (e.g. ultrasonic), and/or injection moulding (e.g. twin-shot or over-moulding).

The exemplary embodiments set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

1. A volute for a turbomachine, the volute comprising a scroll, a volute exit, and a tongue between the scroll and the volute exit, the tongue comprising porous material, wherein the porous material is attached to a wall of the volute.

2. The volute for a turbomachine according to claim 1, wherein the porous material is infrangible.

3. The volute for a turbomachine according to claim 1, wherein the porous material forms a leading edge of the tongue.

4. The volute for a turbomachine according to claim 1, wherein the wall divides the scroll and the volute exit and the porous material extends along a side of the wall.

5. The volute for a turbomachine according to claim 1, wherein the porous material extends about a leading edge of the wall and continuously therefrom along opposite sides of the wall.

6. The volute for a turbomachine according to claim 1, wherein the porous material has an open cell structure.

7. The volute for a turbomachine according to claim 1, wherein the wall defines a recess and the porous material is at least partially located in the recess.

8. The volute for a turbomachine according to claim 1, wherein the volute comprises first and second parts, and the porous material is at least partially sandwiched between the first and second parts.

9. The volute for a turbomachine according to claim 1, wherein the porous material is attached to the wall by one or more of an interference fit, adhesive, or a weld.

10. A turbomachine comprising an impeller, an electric motor operable to rotate the impeller, and a volute according to claim 1 for collecting fluid expelled by the impeller.

11. A kit of parts for building a turbomachine, the kit comprising:
an impeller;
an electric motor operable to rotate the impeller; and
a volute according to claim 1 for collecting fluid expelled by the impeller.

12. A wearable air purifier, comprising a turbomachine according to claim 10, headgear for mounting on a wearer's head to attach the turbomachine to a wearer's head, and a duct for directing fluid discharged by the turbomachine towards a wearer's face when the headgear is mounted on the wearer's head.

13. A method of forming a volute for a turbomachine, the method comprising providing a volute precursor comprising a scroll and a volute exit, and attaching porous material to a wall of the volute precursor to form a tongue between the scroll and the volute exit.
